SEQUENCE LISTING (26 sequences)

SEQ ID NO: 1 - DNA, 29 bp, Artificial
  Synthetic Primer
agaggtacccgttttaagagcttggtgag

SEQ ID NO: 2 - DNA, 31 bp, Artificial
  Synthetic primer
agaggatccctttgccttcgtttatcttgcc

SEQ ID NO: 3 - DNA, 29 bp, Artificial
  Synthetic primer
agagtcgaccgttttaagagcttggtgag

SEQ ID NO: 4 - DNA, 31 bp, Artificial
  Synthetic primer
agactcgagctttgccttcgtttatcttgcc

SEQ ID NO: 5 - DNA, 39 bp, Artificial
  Synthetic primer
agaagatctataatgactaaatctcattcagaagaagtg

SEQ ID NO: 6 - DNA, 34 bp, Artificial
  Synthetic primer
agagctagcttataattcatatagacagctgccc

SEQ ID NO: 7 - DNA, 38 bp, Artificial
  Synthetic primer
agagtcgacataatggcagttgaggagaacaatgtgcc

SEQ ID NO: 8 - DNA, 32 bp, Artificial
  Synthetic primer
gagtctagattattctagcatggccttgtacc

SEQ ID NO: 9 - DNA, 29 bp, Artificial
  Synthetic primer
agagagctcaaagctagtattgtagaatc

SEQ ID NO: 10 - DNA, 32 bp, Artificial
  Synthetic primer
agaggatccttatattgaattttcaaaaattc

SEQ ID NO: 11 - DNA, 34 bp, Artificial
  Synthetic primer
agaagatctataatgaagctactgtcttctatcg

SEQ ID NO: 12 - DNA, 32 bp, Artificial
  Synthetic primer
agagctagcttactctttttttgggtttggtg

SEQ ID NO: 13 - DNA, 31 bp, Artificial
  Synthetic primer
gaatctagaacaggccccttttcctttgtcg

SEQ ID NO: 14 - DNA, 27 bp, Artificial
  Synthetic primer
agagtcgacgttacatgcgtacacgcg

SEQ ID NO: 15 - DNA, 31 bp, Artificial
  Synthetic primer
gaagctagcacaggccccttttcctttgtcg

SEQ ID NO: 16 - DNA, 27 bp, Artificial
  Synthetic primer
agagtcgacgttacatgcgtacacgcg

SEQ ID NO: 17 - DNA, 31 bp, Artificial; identical to SEQ ID NO: 13
  Synthetic primer

SEQ ID NO: 18 - DNA, 27 bp, Artificial
  Synthetic primer
agaggtaccgttacatgcgtacacgcg

SEQ ID NO: 19 - DNA, 30 bp, Artificial
  Synthetic primer
agagtcgacgattcggtaatctccgaacag

SEQ ID NO: 20 - DNA, 35 bp, Artificial
  Synthetic primer
agagagctcgggtaataactgatataattaaattg

SEQ ID NO: 21 - DNA, 41 bp, Artificial
  Synthetic primer
agagtcgacaacgacattactatatatataatataggaagc

SEQ ID NO: 22 - DNA, 30 bp, Artificial
  Synthetic primer
agagagctcaggcaagtgcacaaacaatac

SEQ ID NO: 23 - DNA, 40 bp, Artificial
  Synthetic primer
agagagctctcgaggagaacttctagtatatctacatacc

SEQ ID NO: 24 - DNA, 33 bp, Artificial
  Synthetic primer
agagagctctcgactacgtcgtaaggccgtttc

SEQ ID NO: 25 - DNA, 36 bp, Artificial
  Synthetic primer
agaagatctataatggctagcaaaggagaagaactc

SEQ ID NO: 26 - DNA, 32 bp, Artificial
  Synthetic primer
gagcctaggtcagttgtacagttcatccatgc

(19) United States
(12) Patent Application Publication
(10) Pub. No.: US 2007/0072267 A1
(43) Pub. Date: Mar. 29, 2007

(54) Transformant having galactose induction system and use thereof

(75) Inventors: Takashi Matsuyama, Aichi-gun (JP); Haruo Takahashi, Ohgaki-shi (JP); Kenro Tokuhiro, Aichi-gun (JP); Nobuhiro Ishida, Aichi-gun (JP); Eiji Nagamori, Nisshin-shi (JP)

Correspondence Address: OLIFF & BERRIDGE, PLC, P.O. BOX 19928, ALEXANDRIA, VA, 22320, US

(73) Assignee: KABUSHIKI KAISHA TOYOTA CHUO KENKYUSHO, AICHI-GUN (JP)

(21) Appl. No.: 11/529,443

(22) Filed: Sep. 29, 2006

(30) Foreign Application Priority Data
Sep. 29, 2005 (JP) 2005-285048

(51) Int. Cl. C07K 14/47; C07H 21/04; C12P 21/06; C12N 1/18; C12N 15/74

(52) U.S. Cl. 435/69.1; 435/483; 435/254.2; 530/350; 536/23.5

(57) ABSTRACT

The present invention provides a transformant having a gene expression inducing system having better responsiveness. The transformant of the invention has a galactose induction system and retains a coding region having a galactose induction system and encoding an assisting protein assisting transcription activation by a protein having the Gal4 activity under control of a constitutive promoter, and a coding region encoding an endogenous or exogenous desired protein under control of a galactose-inducible promoter.

### BACKGROUND OF THE INVENTION

1. Field of the Invention

The present invention relates to gene expression technique using a galactose induction system, more particularly, a transformant, a nucleic acid construct, an expression vector, a culturing method, a gene expression method, a substance producing method and the like.

2. Description of the Related Art

Previously, it has been desired to induce gene expression at a desired timing in a cell or the like. For example, in fermentation production using a transformant, maximum proliferation of a cell and, thereafter, production of a desired gene product is preferable in energy balance and a recovery procedure of a gene product. Generally, examples of an element which can be utilized in such the gene expression induction include deficiency of glucose and supply of a new substrate. Glucose is a nutrient which is most easily utilized to many organisms, and various responses are easily generated due to deficiency of glucose in an organism.

As response at such the glucose deficiency, there is galactose induction in budding yeast, Saccharomyces cereivisiae or the like. Galactose induction is a system in which a galactose metabolism gene (GAL gene) group is activated and induced by galactose at the time of glucose deficiency. In galactose induction, a GAL gene group associated with galactose metabolism is positively controlled by a Gal4 protein (hereinafter, simply referred to as Gal4) which is only one transcription activating factor and, when galactose is not present in a medium, initiation of transcription of a GAL gene group is suppressed, but when glucose is not present in a medium and galactose is added, this suppression is eliminated, and expression of a GAL gene group is induced.

It is said that Gal4 activates respective genes of a GAL1 (galactokinase) gene, a GAL2 (galactopermease) gene, a GAL7 (a-D-galactose-1-phosphate uridyltransferase) gene and a GAL10 (uridinephosphogalactose-1-epimerase) gene (hereinafter, simply referred to as GAL10, etc.) necessary for galactose metabolism in budding yeast or the like and, additionally, activates GAL80, GAL3 and MEL1 (D. Lohr et al, The FASEB Journal (1995), p. 777-787).

Japanese Patent Publication No. 2708154 discloses ligation of a Gal4 gene under control of a galactose-inducible promoter and incorporation into a chromosome of budding yeast utilizing the transcription activating ability of such the Gal4 GAL gene group. That is, in this prior art, it is intended that, in such the recombinant yeast, an amount of glucose and an amount of galactose in a medium are controlled to overexpress Gal4.

### SUMMARY OF THE INVENTION

In the procedure of the prior art, in view of that an amount of Gal4 as a positive transcription regulating factor is very small, it is aimed that expression of a desired foreign gene ligated to other many galactose-inducible promoters is effectively performed by inducing Gal4 by galactose to overexpress it.

However, when such the Gal4 is galatose-inducibly overexpressed, a foreign gene is not necessarily galactose-inducibly expressed as intended. Its responsiveness is not sufficient, such as necessity of the same extent of galactose which is a reaction substrate as that of glucose, and a necessary long reaction time.

An object of the present invention is to provide a transformant having a gene expression inducing system which has better responsiveness, and use thereof. That is, one object of the present invention is to provide a transformant for inducing gene expression having sensitive responsiveness, a nucleic acid construct, an expression vector, a culturing method, a gene expression method, and a substance producing method. In addition, another object of the present invention is to provide a transformant for inducing gene expression having rapid responsiveness, a nucleic acid construct, an expression vector, a culturing method, a gene expression method, and a substance producing method.

The present inventors studied a galactose induction system in detail and, as a result, found out that, in a galactose induction system, by constitutively expressing a protein involved in elimination of suppression of the Gal4 activity with Gal80 under control of Gal4 or a protein involved in intake of galactose rather than controlling Gal4 itself, a galactose induction system is manifested at better responsiveness to galactose, which resulted in completion of the present invention. That is, according to the present invention, the following means is provided.

The present invention provides a transformant having a galactose induction system, and retaining a coding region encoding an assisting protein which assists transcription activation by a protein having the Gal4 activity under control of a constitutive promoter on a chromosome or extrachromosomally.

In the transformant of the invention, the assisting protein can include a protein having a Gal80 binding activity and/or a galactokinase activity. In this aspect, the assisting protein may be selected from a protein having the Gal1 activity and a protein having the Gal3 activity. Further, the protein having the Gal80 binding activity may be a protein having the Gal1 activity.

In the transformant of the invention, the assisting protein can include a protein having the galactose transporter activity, and the assisting protein may be a protein having the Gal2 activity.

In the transformant of the invention, further, it is preferable that the assisting protein includes a protein having the Gal80 binding activity and/or the galactokinase activity and a protein having the galactose transporter activity. It is preferable that the assisting protein includes a protein having the Gal1 activity and a protein having the Gal2 activity.

It is preferable that the constitutive promoter is selected from a HIS3 promoter, a TDH3 promoter and an ADH1 promoter.

In addition, it is preferable that a coding region encoding a protein having the Gal4 activity under control of a galactose-inducible promoter is retained on a chromosome or extrachromosomally. In addition, it is preferable that a coding region encoding an endogeneous or exogeneous desired protein is retained under control of a galactose-inducible promoter. In this case, it is preferable that the galactose-inducible promoter is selected from a group consisting of a GAL1 promoter, a GAL2 promoter, a GAL3 promoter, a GAL4 promoter, a GAL5 promoter, a GAL7 promoter, a GAL10 promoter and a MEL1 promoter. Further, the galactose-inducible promoter may be an endogeneous promoter or an exogeneous promoter other than a galactose metabolism gene which is provided with a Gal4 binding site on an upstream site can be activated by a protein having the Gal4 activity. The exogeneous promoter or the endogeneous promoter can be selected from a CYC1 promoter, a CUP1 promoter and a HOR7 promoter.

It is preferable that the aforementioned host cell is yeast, and yeast can be selected from Saccharomyces cerevisiae, Saccharomices pombe, Pichia pastris, Kluyveromyces lactis and Candida albicans.

The present invention also provides a nucleic acid construct comprising a constitutive promoter, and a coding region being ligated under control of the constitutive promoter and encoding an assisting protein which assists transcription activation by a protein having the Gal4 activity.

In the nucleic acid of the invention, it is preferable that the assisting protein includes a protein having the Gal80 binding activity and/or the galactokinase activity, and the assisting protein can be selected from a protein having the Gal1 activity and a protein having the Gal3 activity.

In the nucleic acid of the invention, it is preferable that the assisting protein includes a protein having the galactose transporter activity, and the assisting protein can be a protein having the Gal2 activity.

The present invention also provides another nucleic acid construct comprising a first constitutive promoter, a first coding region ligated under control of the first constitutive promoter and encoding a protein having the Gal80 binding activity and/or a protein having the galactokinase activity, a second constitutive promoter, and a second coding region ligated under control of the second constitutive promoter and encoding a protein having the galactose transporter activity.

The present invention further provides an expression vector comprising any one of the aforementioned nucleic acid constructs. The vector may be a plasmid or a virus. In addition, the present invention further provides a host cell retaining the expression vector.

The present invention further provides a culturing method comprising a culturing step of culturing any one of the aforementioned transformants in a medium not substantially containing galactose. The culturing method of the invention may further comprise a culturing step of culturing the transformant substantially in the absence of glucose, and in the presence of galactose to such an extent that the galactose-inducible system of the transformant can be expressed.

The present invention further provides a method of expressing a gene comprising a first culturing step of culturing any one of the aforementioned transformants retaining a coding region encoding an endogenous or exogeneous desired protein under control of a galactose-inducible promoter in a medium not substantially containing galactose, and a second culturing step of culturing the transformant substantially in the absence of glucose and in the presence of galactose to such an extent that the galactose induction system of the transformant can be expressed to express the desired protein.

In the method of expressing a gene of the invention, it is preferable that an initial concentration of galactose in the second culturing step is less than 20 g/l. In addition, the second culturing step can be initiated by exchanging the medium with a medium containing galactose.

The present invention still further provides a process for producing a substance using a transformant, comprising a step of recovering a desired protein expressed by the aforementioned gene expression method or a metabolite of the protein.

### BRIEF DESCRIPTION OF THE DRAWINGS

FIG. 1 is a view showing a relationship between galactose, Gal80 Gal4 and an assisting protein in a galactose induction system;

FIG. 2 is a view showing pBS-HIS3p-GAL1-HIS3p-GAL2;

FIG. 3 is a view showing pBS-GAL10p-GAL4;

FIG. 4 is a view showing pBS-GAL1p-GFP;

FIG. 5 is a view showing a transformed strain 1;

FIG. 6 is a view showing a transformed strain 2;

FIG. 7 is a view showing a transformed strain 3;

FIG. 8 is a view showing a transformed strain 4;

FIG. 9 is a view showing a transformed strain 5;

FIG. 10 is a view showing a transformed strain 6;

FIG. 11 is a view showing a transformed strain 7;

FIG. 12 is a view showing a transformed strain 8;

FIG. 13 is a view showing image data of a membrane of western blotting in Example 3;

FIG. 14 is a view showing a graph of image data of FIG. 13; and

FIG. 15 is a view showing image data of a membrane of western blotting in Example 3.

### BEST MODE FOR CARRYING OUT THE INVENTION

Various aspects of the transformant, the nucleic acid construct, the vector, the method of culturing a cell, and the process for producing a substance of the present invention intend to constitutively express an assisting protein which assists transcription activation by a protein having the Gal4 activity (hereinafter, simply referred to as Gal4 activity protein). Various aspects of the present invention are based on finding that, by constantly expressing such the assisting protein, a galactose induction system can be induced responsively.

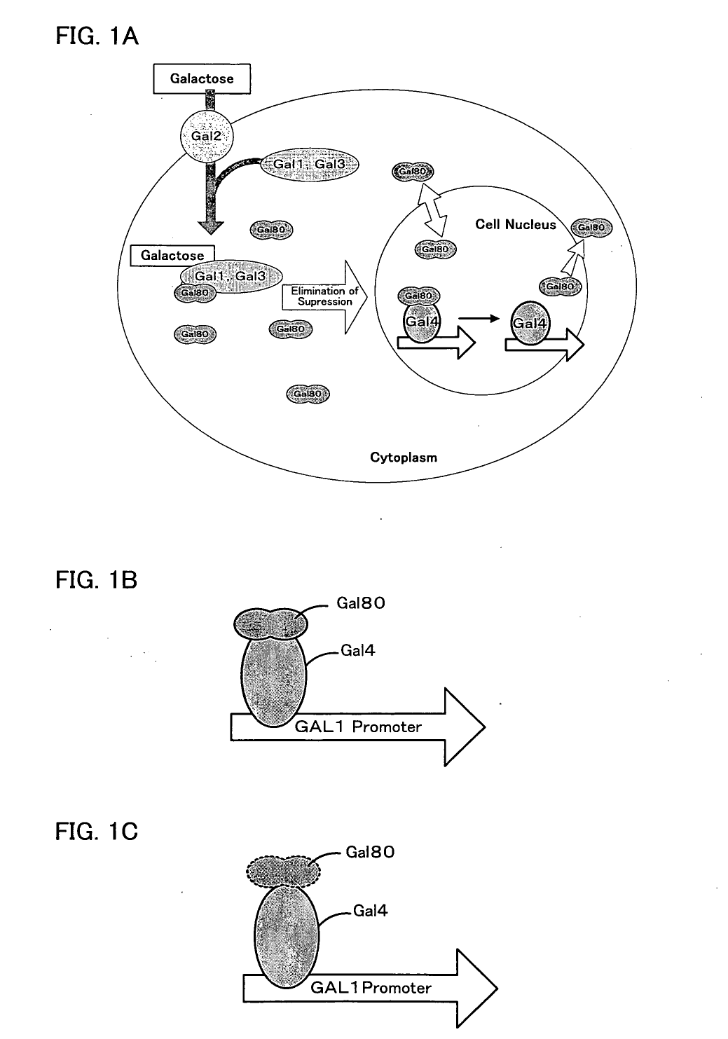

FIG. 1 shows an outline of a relationship between galactose, Gal4 and various assisting proteins among the controlling mechanism contemplated in a galactose induction system. As shown in FIG. 1A, Gal4, as a transcription activating factor of a GAL gene group, exerts the transcription activating action by binding of Gal4 to a UAS gal site on an upstream side of a GAL promoter such as GAL1. As shown in FIG. 1B, when galactose is not present in a medium, since Gal80 which is an inhibiting factor binds to Gal4, transcription can not be activated, and expression of GAL1 is suppressed. On the other hand, as shown in FIG. 1C, when glucose is not present in a medium, and galactose is present in a medium, the inhibiting action of Gal80 is eliminated, and transcription of GAL1 is activated. In addition, although it is known that Gal1 or Gal3 bound to galactose can bind to Gal80 not the all functions of Gal1 or Gal3 have been elucidated. In addition, galactose in a medium is taken in a cell by Gal2 having the galactosepermease activity, and Gal1 has the galactokinase activity which is an enzyme at a first stage of galactose metabolism. However, a relationship therebetween has not also been elucidated.

The present inventors found out that, by constitutively expressing an assisting protein such as a protein having the Gal80 binding activity and/or the galactokinase activity, a galactose-inducible promoter can be operated rapidly and at a high sensitivity exceeding expectation, in the presence of galactose. For this reason, in the absence of galactose, a transformant can be sufficiently proliferated without expressing a gene under control of a galactose-inducible promoter, for example, a protein heterogeneous in quality and/or quantity such as a foreign protein, that is, without expressing a gene which may inhibit proliferation. And, thereafter, a gene under control of a galactose-inducible promoter can be expressed at once and at a large amount galactose-inducibly, in a transformant proliferated at a high density or at a large scale volume. According to the present invention, a galactose induction system can be induced more responsively than inducible overexpression of Gal4.

In addition, in the present specification, for example, when a protein is expressed by adding an active protein to a name of a protein such as Gal4 active protein, in addition to a protein relating to the name, a protein other than a protein relating to the name, which has the physiological activity necessary in the present invention among the physiological activities of a protein relating to the name is also included. Therefore, a protein which is obtained by modifying a natural protein relating to the name or is artificially synthesized is included as far as it has such the physiological activity. In addition, a protein corresponding to Gal4 in a galactose induction system in a cell other than Saccharomyces cerevisiae is also included. In addition, in order to alter a protein, information on a physiologically active site in a natural protein is necessary. Information of a physiologically active site can be specified using a procedure such as insertion of site-specific mutation and the like. Alternatively, the protein can be also obtained by retrieving a homolog, an ortholog, paralog of a natural protein using the known homology search system, and specifying a potential physiologically active site between these proteins.

The transformant, the nucleic acid construct, the vector, the culturing method and the process for producing a substance using a transformant of the present invention will be explained sequentially.

(Transformant)

(Galactose Induction System)

The transformant of the present invention has a galactose induction system. In the present specification, a galactose induction system means a gene group involved in galactose metabolism which is transcription-activated by galactose as a nutrient source. As a galactose induction system, for example, a GAL gene group possessed by Saccharomyces cerevisiae yeast is known. Examples of a GAL gene group in Saccharomyces cerevisiae include GAL1, GAL2, GAL5, GAL7, and GAL10 which are positively controlled by GAL4, and Gal4 which are a gene product of GAL4. In addition, examples include GAL80, GAL3 and MEL1 which are similarly controlled positively by Gal4. In addition, a component constituting a galactose induction system includes a regulating region such as a promoter and its Gal4 binding site in addition to these structural genes.

It is preferable that a galactose induction system possessed by a transformant is a galactose induction system originally possessed by a host cell. That is, as a host cell, a cell having a galactose induction system is used. Such the cell may be a cell provided with an equivalent galactose induction system, and may be other species such as Saccharomyces pombe, S. carlsbergensis, S. norbensis, S. diastaticus, S. oviformis, S. uvarum, S. rouxii, S. montanus, S. kluyveri, S. elengisporus and the like in addition to Saccharomyces cerevisiae. Also, the cell may be of other genera such as Pichia and Kluyveromyces. Further, the cell may be Candida genus such as Candida albicans. By using a cell provided with a galactose induction system as a host cell, the number of foreign genes to be introduced into a transformant can be suppressed.

In addition, a galactose induction system possessed by a transformant may be a galactose induction system comprising an endogeneous gene of a host cell, and a part or all of it may be artificially constructed. That is, as a host cell, in addition to a cell originally having a galactose induction system, a cell in which a galactose induction system is constructed by a genetic engineering procedure can be used. For example, a foreign gene group which can construct a galactose induction system may be introduced into a host cell having no galactose induction system, or in the case where a cell has the galactose metabolizing ability but is not galactose inducing, a cell may be altered to be galactose inducing, or a part or all of a structural gene or a regulating region of a galactose induction system of a host may be substituted with a foreign gene. In addition, a component of a galactose induction system may be such that an endogeneous structural gene or a transcription regulating region may be altered, or may be deleted as far as galactose metabolism is possible in a galactose inducing manner. For example, Gal4 or Gal80 in a galactose induction system of Saccharomyces cerevisiae may be altered so as to express a Gal4 active protein or a Gal80 active protein. In addition, it is not necessary that an entire galactose induction system is on a host chromosome, but it is enough that the system is functionally retained in a host chromosome and/or extrachromosomally.

From the forgoing, as a host cell of the present invention, a unicellular eukaryote such as yeast is preferable. Preferable examples of such the host cell include, among others, Saccharomyces cerevisiae, Saccharomyces pombe, Candida albicans, Pichia pastris, and Kluyveromyces lactis. More preferable is Saccharomyces cerevisiae, and Kluyveromyces lactis.

(Constitutive Promoter)

A transformant retains a coding region encoding a transcription activation assisting protein by a Gal4 active protein under control of a constitutive promoter. Herein, constitutive means regardless of the-growing condition of a host cell, and a constitutive promoter means a promoter which expresses a gene under control regardless of the growing condition of a host cell. A constitutive promoter is not particularly limited, but may be appropriately selected depending on a kind of a host cell used or depending on a kind of a gene to be controlled. For example, examples of a constitutive promoter in Saccharomyces cerevisiae include an ADH1 promoter, a HIS3 promoter, a TDH3 promoter, a CYC3 promoter, a CUP1 promoter and a HOR7 promoter. In the present specification, when there is the description of an ADH1 promoter, a variant consisting only of a part of a nucleotide sequence of this promoter, and various variants such as a variant in which a part of a nucleotide sequence is substituted, deleted, or inserted are included. As a constitutive promoter, a promoter at an expression level depending on a kind of an assisting protein can be selected. For example, as a constitutive promoter of a protein having the Gal80 binding activity and/or the galactokinase activity, a HIS3 promoter or a CYC1 promoter can be selected. In addition, as a constitutive promoter of a protein having the galactose transporter activity such as a Gal2 active protein, a HIS3 promoter can be used. A HIS3 promoter is preferable in an aspect in which a galactose transporter active protein is expressed at the same time with a protein having the Gal80 binding activity and/or the galactokinase activity.

(Assisting Protein which Assists Transcription Activation by Gal4 Active Protein)

In the present invention, an assisting protein which assists transcription activation by a Gal4 active protein may be a protein other than a Gal4 active protein, which consequently eliminates the transcription activation inhibiting action of a Gal80 active protein on a Gal4 active protein. In addition, a Gal4 active protein may be specifically a protein which binds to a Gal4 binding site or UASgal on a chromosome and, at the same time, positively controls transcription activity of a downstream gene by interaction with a Gal80 active protein or the like. Therefore, the protein may be a protein which has been artificially altered while binding activity to a Gal4 binding site and transcription control activity are maintained, in addition to Gal4 of Saccharomyces cerevisiae. Alternatively, the protein may be a protein of yeast of other species or a genus corresponding to Gal4. A full amino acid sequence of Gal4 (Genbank accession number: Z73604), and a DNA binding active site, a transcription activation site and the like are already known (D. Lohr et al, The FASEB Journal (1995), p. 777-787), and a person skilled in the art can easily prepare an artificial protein based on such the sequence.

In the present invention, it is preferable that an assisting protein is a protein having the Gal80 binding activity and/or the galactokinase activity. It is considered that a protein having the Gal80 binding activity can eliminate the transcription activation inhibiting action of a Gal8O active protein on a Gal4 active protein. In addition, it is considered that a protein having the galactokinase activity promotes intake of galactose into a cell, and it is considered that the binding activity of a protein having the Gal80 binding activity is promoted. In addition, the Gal80 binding activity is binding activity on a Gal80 active protein, which consequently eliminates the action of inhibiting of transcription of a galactose induction system, of a Gal80 active protein. In addition, a Gal80 active protein may be a protein which has been artificially altered while the activity of binding to Gal4 to inhibit its transcription activation action is maintained, in addition to Gal80 (Genbank accession number: X01667) of Saccharomyces cerevisiae. In addition, the protein may be a protein of yeast of other species or genus corresponding to Gal80.

As a protein having the Gal80 binding activity and the galactokinase activity, a Gal1 active protein can be used. In addition, as a protein having the Gal80 binding activity, a Gal1 active protein and a Gal3 active protein can be used. As a protein having the galactokinase activity, a Gal1 active protein can be used. In a galactose induction system of Saccharomyces cerevisiae, it is known that Gal1 and Gal3 have the ability to bind to Gal80 and promote elimination of the transcription activation inhibiting action of Gal80 on Gal4. In addition, Gal1 has the galactokinase activity.

As a Gal1 active protein, in addition to Gal1 (Genbank accession number: Z35889), any of a protein having the Gal1 galactosekinase activity and the Gal80 binding activity, a protein having the galactokinase activity and a protein having the Gal80 binding activity can be used. Preferably, Gal1, and a protein having the galactokinase activity and the Gall80 binding activity can be used. As a Gal3 active protein, in addition to Gal3, a protein having the Gal80 binding activity of Gal3 can be used. In addition, a Gal1 active protein may be, in addition to Gal1 of Saccharomyces cerevisiae, a protein which has the Gal80 binding activity and/or the galactokinase activity, and can eliminate the transcription activation inhibiting action of a Gal80 active protein on a Gal4 active protein, and may be an artificially constructed or altered protein. In addition, the protein may be a protein of yeast of other species or genus corresponding to Gal1. In addition, a Gal3 active protein may be, in addition to Gal3 of Saccharomyces cerevisiae (Genbank accession number: Z74035), a protein which has the Gal80 binding activity, and can eliminate the transcription activation inhibiting action of a Gal80 active protein on a Gal4 active protein, and may be an artificially constructed or altered protein. In addition, the protein may be a protein of yeast of other species or genus corresponding to Gal3.

In addition, as an assisting protein, a protein having the galactose transporter activity can be used. This is because it is considered that the galactose transporter can promote intake of galactose into a cell, and promotes the binding activity of a protein having the Gal80 binding activity. As a protein having the galactose transporter activity, a Gal2 active protein can be used. It is known that a Gal2 active protein is degraded in a cell in the presence of glucose. For this reason, in order to express a galactose induction system responsively using this assisting protein alone, it is preferable to constitutively express the system at a level equivalent to or higher than the case where used with a protein having the Gal80 binding activity, and it is preferable to select such the promoter. As a Gal2 active protein, in addition to Gal2 (Genbank accession number: Z73253), a protein having the galactose transporter activity of Gal12 can be used. In addition, a Gal2 activity protein may be, in addition to Gal2 of Saccharomyces cerevisiae, a protein having the galactose transporter activity, and may be an artificially constructed or modified protein. In addition, the protein may be a protein of yeast of other species or genus corresponding to Gal2.

In the present invention, it is preferable to use a protein having at least the Gal80 binding activity and/or the galactokinase activity as an assisting protein, and it is more preferable to use a protein having the Gal80 binding activity and the galactokinase activity. That is, a Gal1 active protein or a Gal3 protein can be preferably used, and a Gal1 active protein can be used more preferably. Although, the present invention in not constrained, it is presumed that, by constitutively expressing a protein having the Gal80 binding activity and the galactokinase activity, the protein promotes intake of galactose into a cell, at the same time, binds with taken galactose to exert the Gal80 binding activity in a cytoplasm, and can rapidly reduce a concentration of a Gal80 active protein in a cell nucleus. It is presumed that, by constitutively expressing either of a protein having the Gal80 binding activity or a protein having the galactokinase activity, a concentration of a Gal80 active protein in a cell nucleus can be rapidly reduced, and a galactose induction system can be induced responsively.

In addition, it is preferable to use, as an assisting protein of the present invention, a Gal80 binding active protein and/or a protein having the galactokinase activity together with a protein having the galactose transporter activity. That is, it is preferable to use a Gal2 active protein with a Gal1 active protein and/or a Gal3 active protein, and it is preferable to use a Gal1 active protein and a Gal2 active protein. Although, the present invention is not constrained, it is presumed that, by constitutively expressing a protein having the galactose transporter activity, a galactose transporter active protein is rapidly retained by a cell membrane accompanied with deficiency in glucose, intake of galactose is promoted, a concentration of a Gal80 active protein in a cell nucleus is reduced, and a galactose induction system can be induced responsively.

A tranformant retains a coding region encoding such the assisting protein. A coding region can be determined on the basis of the known amino acid sequences and nucleotide sequences of various assisting proteins, and can be also determined on the basis of information on various active sites of various proteins of a galactose induction system, and results of determination of an activating site by a procedure such as site-specific mutation introduction and the like. A polynucleotide of a coding region is preferably a DNA, and a DNA may include a c intron, and is preferably a cDNA. In addition, a coding region may contain an artificial deoxyribonucleotide, or may be modified. In addition, a constitutive promoter and a coding region may be on a host chromosome or outside a chromosome. Preferably, they are retained on a host chromosome.

(Gal4 Active Protein)

A transformant may galactose-inducibly express a Gal4 active protein. That is, it may retain a coding region encoding a Gal4 active protein under control of a galactose-inducible promoter. Even when a Gal4 active protein is galactose-inducibly expressed without constitutively expressing an assisting protein, better responsiveness has not been obtained, but by constitutively expressing an assisting protein and, thereafter, galactose-inducibly expressing a Gal4 active protein, responsiveness of induction of a galactose induction system by a constitutively expressed assisting protein is enhanced, and an expression intensity can be potentiated.

As the galactose-inducible promoter, promoters of respective genes of GAL1, GAL2, GAL3, GAL5, GAL7, GAL10 and MEL1 can be used. In the present description, such the promoter may be altered as far as it is galactose-inducible, and such the altered promoter is also included, as described above. In addition, these promoters contain a Gal4 active protein-binding site such as UASgal. Preferably, any promoter of GAL1, GAL2, GAL7 and GAL10 is used and, more preferably, a GAL10 promoter is used.

In addition, an endogeneous promoter or an exogeneous promoter other than a galactose metabolism gene, which is provided with a Gal4 binding site on an upstream site, and can be activated by a protein having the Gal4 activity can be used as a galactose-inducible promoter. In addition, it is well-known that even a promoter which is not a so-called galactose-inducible promoter functions as a galactose-inducible promoter by having a Gal4 binding site on its upstream site. For example, by retaining a Gal4 binding site on an upstream site of a promoter originally possessed by a host cell, the endogeneous promoter can be utilized as a galactose-inducible promoter. This is also true in the case of an exogeneous promoter which is not galactose-inducible. As such the endogeneous promoter and the exogeneous promoter, a CYC1 promoter, a CUP1 promoter, and a HOR7 promoter can be preferably used.

A region for galactose-inducibly expressing a Gal4 active protein may be retained on a host chromosome, or may be retained outside a host chromosome, and is preferably retained on a host chromosome.

The above-explained tranfomant of the present invention is provided with an altered galactose induction system, suitable for galactose-inducibly expressing a desired gene responsively. That is, in such the transformant, by expressing a desired gene under control of a galactose-inducible promoter, a transformant suitable for efficient substance production can be obtained. Therefore, such the transformant can be used as a preferable host cell for expressing a desired gene under control of a galactose induction system.

(Transformant Retaining Coding Region Encoding Endogeneous or Exogeneous Desired Protein under Control of Galactose-Inducible Promoter)

The transformant of the present invention can be a transformant which galactose-inducibly expresses an endogeneous or exogeneous desired protein. That is, a coding region encoding an endogeneous or exogeneous desired protein under control of a galactose-inducible promoter can be retained. According to this transformant, since responsiveness of a galactose induction system is improved, a gene can be galactose-inducibly expressed responsively to obtain a desired protein or a metabolite thereof. A galactose-inducble promoter can be used by appropriately selecting it from the already explained promoters. Preferably, a GAL1 promoter is used.

A desired protein to be galactose-inducibly expressed is preferably other than a protein involved in galactose metabolism, and is selected depending on necessity. For example, various fluorescent proteins such as GFP for visualizing an expression level in study utility or the like can be used. A region for galactose-inducibly expressing such the desired protein may be either on a host chromosome or outside a host chromosome, and is preferably on a host chromosome.

(Process for Producing Transformant)

Such the transformant can be obtained, preferably, by introducing a nucleic acid construct provided with an expression cassette containing a coding region of a protein having the Gal binding activity, which is connected under control of a constitutive promoter, into a host cell having a galactose induction system using the known gene introducing method such as a transformation method, a transfection method, a joining method, protoplast fusion, an electroporation method, a lipofection method, a lithium acetate method, a particle gun method. A transformant is selected by a suitable selection medium, and a transformant for which introduction of a gene can be confirmed, can be used.

(Nucleic Acid Construct)

The nucleic acid construct of the present invention comprises a constitutive promoter, and a coding region encoding an assisting protein assisting transcription activation by a protein having the Gal4 activity, which is ligated under control of the constitutive promoter. As the constitutive promoter, the same promoter as that already explained can be used. In addition, as the assisting protein, the assisting protein already explained can be used. Further, as a transcription regulating region, in addition to a terminator, if necessary, a cis element such as an enhancer, a splicing signal, a poly A addition signal, a selectable marker, and a ribosome binding sequence (a SD sequence) can be ligated. As the selectable marker, the known various selectable marker genes such as a drug resistance gene, and an auxotrophy gene can be utilized without any limitation.

The nucleic acid construct may encode one kind of an assisting protein, or may encode two or more kinds of assisting proteins. For example, the nucleic acid construct may comprise a first constitutive promoter, a first coding region encoding a first assisting protein such as a protein having the Gal80 binding activity and/or the galactokinase activity, which is ligated under control of the first constitutive promoter, a second constitutive promoter, and a second coding region encoding a second assisting protein such as a protein having the galactose transporter activity, which is ligated under control of the first constitutive promoter. The nucleic acid construct may comprise one or more homologous regions with a host chromosome, and may be constructed as a chromosome introduction-type.

Various components in the nucleic acid construct can be obtained by the previously known procedure such as a PCR method, and such the nucleic acid construct is retained in a suitable plasmid vector or the like. Depending on a gene introducing method used, a construct is excised with a restriction enzyme or the like at a necessary time, and the construct itself is used. Obtaining of a component, and a procedure of constructing a construct can be according to the previously known procedure. For example, they may be according to Molecular Cloning A Laboratory Manual second edition (Maniatis et al., Cold Spring Harbor Laboratory Press, 1989).

(Expression Vector)

Such the nucleic acid construct is provided in the form of various vectors. As the vector, a plasmid, a virus, bacteriophage, transposon, and an artificial chromosome (YAC, BAC, PAC, etc.) are selected depending on an aspect of introducing an exogeneous gene (on a chromosome or outside a chromosome), and a kind of a host cell. In addition, such the expression vector is provided in the state where it is retained in a suitable host cell.

(Method of Culturing Transformant)

The method of culturing the transformant of the present invention comprises a culturing step of culturing a transformant which constitutively expresses an assisting protein assisting transcription activation by a protein having the Gal4 activity, and retains a coding region encoding an endogeneous or exogeneous desired protein under control of a galactose-inducible promoter on a chromosome or outside a chromosome of a host cell, in a medium not substantially containing galactose. By having such the culturing step, an exogeneous gene which is galactose-inducibly expressed in a transformant is not expressed, and a cell is proliferated without expressing a protein which is qualitatively and/or quantitatively abnormal to a cell. Thereby, a cell can be proliferated easily, and at a high density or at a large scale. Further, according to this transformant, a galactose induction system can be expressed responsively. Therefore, according to the present culturing method, the cell proliferating state suitable for expressing a galactose induction system sensitively, in a short time and at a large scale can be easily obtained. In the present description, the “medium not substantially containing galactose” includes a medium not containing galactose, and a medium containing galactose at a limitation of such a concentration that a galactose inducing system possessed by a transformant is not induced.

For culturing a transformant, the culturing condition can be selected depending on a host cell and the content of transformation. As a medium for culturing a transformant using yeast as a host, any of a natural medium and a synthetic medium can be used as far as it contains such as a carbon source, a nitrogen source, inorganic salts other than galactose, which can be utilized by yeast, and can effectively culture a transformant. As the carbon source, a carbohydrate such as glucose, fructose, sucrose, starch, cellulose, an organic acid such as acetic acid, propionic acid, and an alcohol such as ethanol, propanol can be used. As the nitrogen source, ammonia, an ammonium salt of an inorganic acid or an organic acid such as ammonium chloride, ammonium sulfate, ammonium acetate, ammonium phosphate and the like, or other nitrogen-containing compound, and peptone, meat extract, corn steep liquor and the like can be used. As the inorganic substance, primary potassium phosphate, magnesium phosphate, magnesium sulfate, sodium chloride, ferrous sulfate, manganese sulfate, copper sulfate, and calcium carbonate can be used.

As the culturing method, the previously known culturing method can be utilized without any limitation. For example, any of batch culturing, semi-batch culturing, continuous culturing and the like, or a combination thereof can be performed. In addition, an oxygen supplying manner, and a stirring manner may be selected depending on a kind of a transformant. In the case of yeast such as Saccharomyces cerevisiae, usually, shaking culturing or aeration stirring culturing is performed under the aerobic condition at 28 to 30° C. for 6 to 24 hours. In addition, during culturing, if necessary, an antibiotic such as ampicillin and tetracycline can be added to a medium.

(Method of Expressing Gene)

The method of expressing a gene of the present invention comprises a first culturing step of culturing a transformant which constitutively expresses an assisting protein assisting transcription activation by a protein having the Gal4 activity, and retains a coding region encoding an endogeneous or exogeneous desired protein under control of a galactose-inducible promoter on a chromosome or outside a chromosome of a host cell, in a medium not substantially containing galactose, and a second culturing step of culturing a transformant in the presence of galactose to such an extent that glucose is not substantially contained, and a galactose induction system can be expressed, to express an endogeneous or exogeneous protein. According to this expression method, by having the first culturing step, as described above, this is in the state suitable for expressing a galactose induction system sensitively, in a short time and at a large scale. Then, by adding galactose to express a galactose induction system in a second culturing step, a galactose induction system is galactose-inducibly expressed at once and at a large scale to express a desired protein galactose-inducibly, thereby, the protein or a metabolite thereof can be obtained.

The first culturing step in the present expression method can be performed in the same various aspects as those of the culturing method of the present invention. In a second culturing step, glucose is not substantially contained. In the present specification, “not substantially containing glucose” means that glucose is not contained, or glucose may be contained at limitation of such an extent of a concentration that induction of a galactose induction system possessed by a transformant is not suppressed.

In addition, an initial concentration of galactose at such an extent that a galactose induction system can be expressed in a second culturing step may be less than 20 g/l, preferably 2 g/l or less, more preferably 1 g/l or less, and further preferably 0.05 g/l or more. In the case of transient galactose induction, it is not particularly necessary that a galactose concentration is maintained, but it is necessary in some cases as needed. In addition, the culturing is preferably performed under the aerobic condition, and a stirring procedure may be performed.

(Process for Producing Gene Product or Secondary Product Thereof)

The process for producing a gene product or a secondary product thereof of the present invention may comprise a step of recovering a desired protein expressed by the method of expressing a gene of the present invention, or a metabolite from the protein. In a step of recovering a protein or a metabolite thereof, for example, when these products are produced in a transformant, the transformant cells are destructed by ultrasound destructing treating, grinding treatment, or pressure destruction by a conventional method and, thereafter, a product and cells can be separated. In this case, if necessary, a protease is added. In addition, when a product is produced outside the cells, this culturing solution is filtered or centrifuged to remove a solid matter such as cell debris. For these crude extraction fractions, these products can be purified utilizing the previously known various purifying and separating methods.

### EXAMPLES

The present invention will be specifically explained below by way of Example, but the present invention is not limited by these Examples at all, but can be implemented in various aspects in a scope without departing from the gist of the present invention.

### Example 1

Construction of Vector

In the present Example, a Saccharomyces cerevisiae-derived galactokinase gene (GAL1 gene) and a galactosepermease gene (GAL2 gene) as an objective gene were used under control of an imidazole glycerol phosphate dehydrogenase gene (HIS3 gene) promoter sequence derived from Saccharomyces cerevisiae. Further, a GAL4 gene derived from Saccharomyces cerevisiae was used under control of a UDP-glucose-4-epimerase gene (GAL10 gene) promoter sequence derived from Saccharomyces cerevisiae. Additionally, a Green Fluorescent Protein gene (GFP gene) derived from Aequorea coerulescens was used under control of a galactokinase gene (GAL1 gene) promoter sequence derived from Saccharomyces cerevisia.

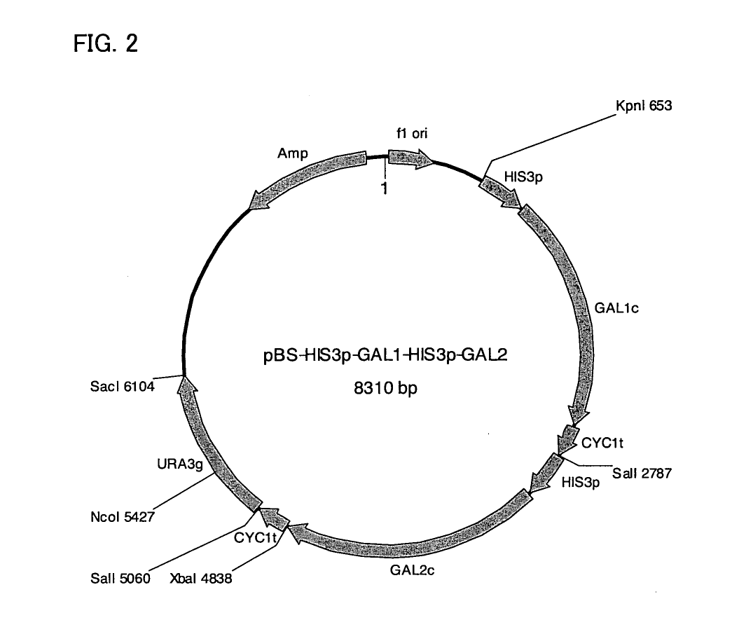

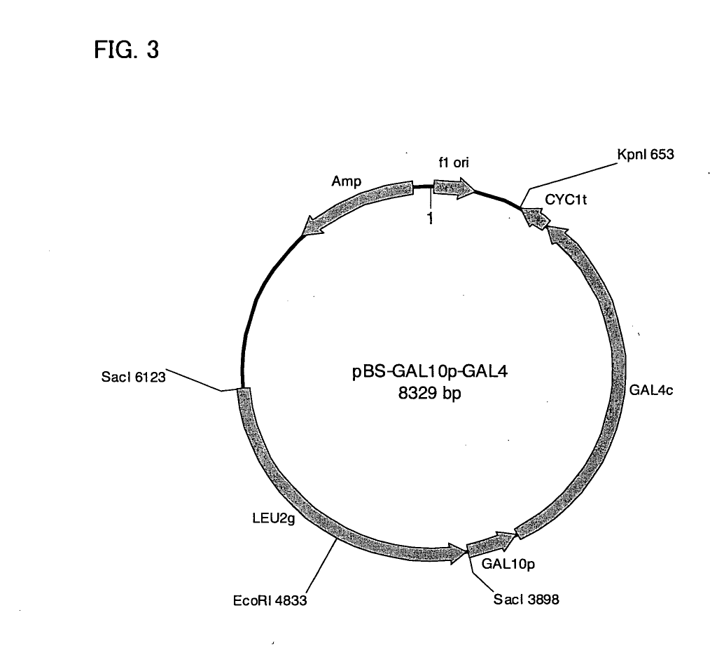

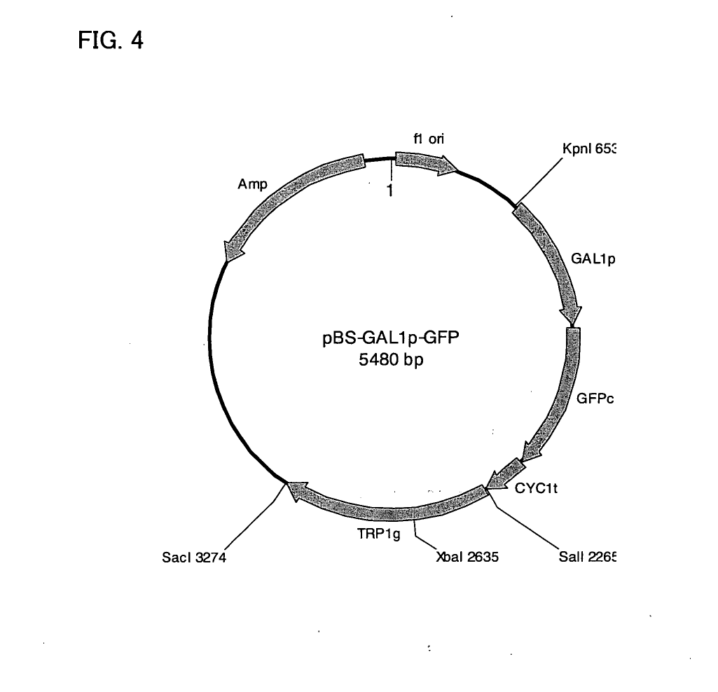

Chromosome introduction-type vectors newly constructed for the present Example were designated as pBS-HIS3p-GAL1-HIS3p-GAL2 (FIG. 2), pBS-GAL10p-GAL4 (FIG. 3), and pBS-GAL1p-GFP (FIG. 4), and details of examples of constructing of the present vectors will be described below. An outline of the present Example will be shown in FIGS. 2 to 4. In this respect, a procedure of constructing vectors is not limited. Upon construction of vectors, a HIS3 gene promoter fragment (HIS3p) 329 bp, a GAL1 gene cDNA fragment (GAL1c) 1608 bp, a GAL2 gene cDNA fragment (GAL2c) 1746 bp, a GAL10 gene promoter fragment (GAL10p) 380 bp, a GAL4 gene cDNA fragment (GAL4c) 2667 bp, and a CYC1 gene terminator fragment (CYC1t) 231 bp which are necessary gene fragments were isolated by a PCR amplifying method employing, as a template, a genomic DNA of Saccharomyces cerevisiae W303-1a strain (ATCC: The Global Bioresourcecenter).

In addition, a URA3 gene fragment (URA3g) 1056 bp was isolated by a PCR amplifying method employing, as a template, a DNA of a plasmid pRS406 (Stratagene), a TRP1 gene fragment (TRP1g) 1021 bp was isolated by a PCR amplifying method employing as a template, a DNA of a plasmid pRS404 (Stratagene), a LEU2 gene fragment (LEU2g) 2237 bp was isolated by a PCR amplifying method employing, as a template, a DNA of a plasmid pRS405 (Stratagene), and a GFP gene cDNA fragment (GFP c) 741 bp was isolated by a PCR amplifying method employing, as a template, a DNA of a plasmid pQBI25 (Wako Pure Chemical Industries, Ltd).

A genomic DNA of Saccharomyces cerevisiae W303-1a strain was prepared using a genome preparation kit Fast DNA Kit (Bio101) according to details of the attached protocol. A DNA concentration was measured with a spectrophotometer Ultrospec 3000 (Amersham Bioscience). In a PCR reaction, as an amplification enzyme, KOD⁺ DNA polymerase (Toyobo) which is said to have high correctness of an amplification fragment was used. A genomic DNA of Saccharomyces cerevisiae W303-1a strain prepared by the aforementioned procedure 50ng/sample, a primer DNA 50 pmol/sample, and KOD⁺ DNA polymerase 0. 2 unit/sample were prepared into a total of 50 μl of a reaction system. The reaction solution was subjected to DNA amplification with a PCR amplifying apparatus gene Amp PCR system 9700 (PE Applied Biosystems). As the reaction condition of a PCR amplification apparatus, after 30 seconds at 94° C., 30 cycles of (30 seconds at 94° C., 30 seconds at 50° C., and 3 minutes at 68° C.) were performed and, thereafter, a temperature was set to be 4° C. HIS3p amplification fragments 1 and 2, a GAL1c amplification fragment, a GAL2c amplification fragment, a GAL10p amplification fragment, a GAL4c amplification fragment, CYC1t amplification fragments 1 to 3, a URA3 amplification fragment, a TRP1 amplification fragment, a LEU2 amplification fragment and a GFPc amplification fragment were subjected to 1% TBE agarose gel electrophoresis to confirm gene amplification fragments. As a primer DNA used in the reaction, synthetic DNAs (Sawaday Technology) were used, and DNA sequences of these primers are as follows.

HIS3p Amplification Fragment 1

A restriction enzyme KpnI site is added to an end of KpnI-His3p (29mer): AGA GGT ACC CGT TTT AAG AGC TTG GTG AG (SEQ ID No.: 1)
A restriction enzyme BamHI site is added to an end of His3-BamHI (31mer): AGA GGA TCC CTT TGC CTT CGT TTA TCT TGC C
   HIS3p Amplification Fragment 2 (SEQ ID No.: 2)
A restriction enzyme SalI site is added to an end of SalI-His3 (29mer): AGA GTC GAC CGT TTT AAG AGC TTG GTG AG (SEQ ID No. : 3)
A restriction enzyme XhoI site is added to an end of His3-XhoI (31mer): AGA CTC GAG CTT TGC CTT CGT TTA-TCT TGC C (SEQ ID No.: 4)
   GAL1c Amplification Fragment
A restriction enzyme BglII site is added to an end of BglII-Gal1c (39mer): AGA AGA TCT ATA ATG ACT AAA TCT CAT TCA GAA GAA GTG (SEQ ID No.: 5)
A restriction enzyme NheI site is added to an end of Gal1c-NheI (34mer): AGA GCT AGC TTA TAA TTC ATA TAG ACA GCT GCC C (SEQ ID No.: 6)
   GAL2c Amplification Fragment
A restriction enzyme SalI site is added to an end of SalI-Gal2c (38mer): AGA GTC GAC ATA ATG GCA GTT GAG GAG AAC AAT GTG CC (SEQ ID No.: 7)
A restriction enzyme XbaI site is added to an end of Gal2c-XbaI (32mer): GAG TCT AGA TTA TTC TAG CAT GGC CTT GTA CC (SEQ ID No.: 8)
   GAL10p Amplification Fragment
A restriction enzyme SacI site is added to an end of SacI-Gal10p (29mer): AGA GAG CTC AAA GCT AGT ATT GTA GAA TC (SEQ ID No.: 9)
A restriction enzyme BamHI site is added to an end of Gal10p-BamHI (32mer): AGA GGA TCC TTA TAT TGA ATT TTC AAA AAT TC (SEQ ID No.: 10)
   GAL4c Amplification Fragment
A restriction enzyme BglII site is added to an end of BglII-Gal4c (34mer): AGA AGA TCT ATA ATG-AAG CTA CTG TCT TCT ATC G (SEQ ID No.: 11)
A restriction enzyme NheI site is added to an end of Gal4-NheI (32mer): AGA GCT AGC TTA CTC TTT TTT TGG GTT TGG TG (SEQ ID No.: 12)
   CYC1t Amplification Fragment 1
A restriction enzyme XbaI site is added to an end of XbaI-Cyc1t (31mer): GAA TCT AGA ACA GGC CCC TTT TCC TTT GTC G (SEQ ID No.: 13)
A restriction enzyme SalI site is added to an end of Cyc1t-SalI (27mer): AGA GTC GAC GTT ACA TGC GTA CAC GCG (SEQ ID No.: 14)
   CYC1t Amplification Fragment 2
A restriction enzyme NheI site is added to an end of NheI-Cyc1t (31mer): GAA GCT AGC ACA GGC CCC TTT TCC TTT GTC G (SEQ ID No.: 15)
A restriction enzyme SalI site is added to an end of Cyc1t-SalI (27mer): AGA GTC GAC GTT ACA TGC GTA CAC GCG (SEQ ID No.: 16)
   CYC1t Amplification Fragment 3
A restriction enzyme XbaI site is added to an end of XbaI-Cyc1t (31mer): GAA TCT AGA ACA GGC CCC TTT TCC TTT GTC G (SEQ ID No.: 17)
A restriction enzyme KpnI site is added to an end of Cyc1t-KpnI (27mer): AGA GGT ACC GTT ACA TGC GTA CAC GCG (SEQ ID No.: 18)
   URA3g Amplification Fragment
A restriction enzyme SalI site is added to an end of SalI-Ura3g (30mer): AGA GTC GAC GAT TCG GTA ATC TCC GAA CAG (SEQ ID No. : 19)
A restriction enzyme SacI site is added to: an end of Ura3g-SacI (35mer): AGA GAG CTC GGG TAA TAA CTG ATA TAA TTA AAT TG (SEQ ID No.: 20)
   TRP1g Amplification Fragment
A restriction enzyme SalI site is added to an end of SalI-5-Trp1g (41mer): AGA GTC GAC AAC GAC ATT ACT ATA TAT ATA ATA TAG GAA GC (SEQ ID No.: 21)
A restriction enzyme SacI site is added to an end of Trp1g-SacI (30mer): AGA GAG CTC AGG CAA GTG CAC AAA CAA TAC (SEQ ID No.: 22)
   LEU2g Amplification Fragment
A restriction enzyme SacI site is added to an end of SacI-Leu2g (40mer): AGA GAG CTC TCG AGG AGA ACT TCT AGT ATA TCT ACA TAC C (SEQ ID No.: 23)
A restriction enzyme SacI site is added to an end of Leu2-SacI (33mer): AGA GAG CTC TCG ACT ACG TCG TAA GGC CGT TTC (SEQ ID No.: 24)
   GFPc Amplification Fragment
A restriction enzyme BglII site is added to an end of BglII-Gfpc (36mer): AGA AGA TCT ATA ATG GCT AGC AAA GGA GAA GAA CTC (SEQ ID No.: 25)
A restriction enzyme ApaI site is added to an end of Gfpc-BlnI (32mer): GAG CCT AGG TCA GTT GTA CAG TTC ATC CAT GC (SEQ ID No.:26)

After respective Amplification Fragments obtained by the aforementioned reaction were purified by ethanol precipitation treatment, restriction enzyme reaction treatment was performed as follows: a HIS3p amplification fragment 1 was treated with a restriction enzyme KpnI/BamHI, a HIS3p Amplification Fragment 2 was treated with a restriction enzyme SalI/XhoI, a GAL1c Amplification Fragment was treated with a restriction enzyme BglII/NheI, a GAL2c amplification fragment was treated with a restriction enzyme SalI/XbaI, a GAL10p Amplification Fragment was treated with a restriction enzyme SacI/BamHI, a GAL4c Amplification Fragment was treated with a restriction enzyme BglII/NheI, a CYC1t amplification fragment 1 was treated with a restriction enzyme XbaI/SalI, a CYC1t Amplification Fragment 2 was treated with a restriction enzyme NheI/Sal1, a CYC1t Amplification Fragment 3 was treated with a restriction enzyme XbaI/KpnI, a URA3 g amplification fragment was treated with a restriction enzyme SalI/SacI, a TRP1 g Amplification Fragment was treated with a restriction enzyme SalI/SacI, a LEU2g Amplification Fragment was treated with a restriction enzyme SacI/SacI, and a GFPc amplification fragment was treated with a restriction enzyme BglII/BlnI, respectively. As enzymes used below, all manufactured by TAKARA HOLDINGS INC. were used. In addition, detailed manual of a series of procedures of ethanol precipitation treatment and restriction enzyme treatment was according to Molecular Cloning A Laboratory Manual second edition (Maniatis et al., Cold Spring Harbor Laboratory Press, 1989).

A series of reaction procedures in construction of a vector were performed according to a general DNA subcloning method. That is, suitable restriction enzyme treatment was performed, and the aforementioned DNA Amplification Fragment was ligated to a pBluescript IISK(−) vector (Stratagene) which had been subjected to dephosphorylation enzyme (Alkaline Phosphatase (BAP, TAKARA HOLDINGS INC.)) by a T4 DNA Ligase reaction. In the T4 DNA Ligase reaction, LigaFast Rapid DNA Ligation System (Promega) was used, and a detail was according to the attached protocol. Then, the Ligation reaction solution was transformed into a competent cell. As the competent cell, an Escherichia coli JM109 strain (Toyobo) was used, and a detail was performed according to the attached protocol. The resulting culturing solution was seeded on a LB plate containing 100 μg/ml of an antibiotic, ampicillin, and this was cultured overnight. The grown colony was cultured overnight in a LB liquid medium (ampicillin 100 μg/ml), a plasmid DNA was prepared with a miniprep, and the solution was confirmed by the restriction enzyme treatment to isolate a strain having an objective construct. The above procedures were repeated to prepare three chromosome introduction-type vectors of pBS-HIS3p-GAL1-HIS3p-GAL2, pBS-GAL10p-GAL4, and pBS-GAL1p-GFP.

As a chromosome introduction-type vector for a control experiment, pBS-HIS3p-GAL1 for testing the effect of only a GAL1 gene, pGS-HIS3p-GAL2 for testing of the effect of only a GAL2 gene, pGS-URA3 g of only an uracil auxotrophic marker, and pBS-LEU2 g of only a leucine auxotrophic marker were also prepared.

For confirming the constructive chromosome introduction-typed vector, a nucleotide sequence was determined. As a nucleotide sequence analyzing apparatus, ABIPRISM 310 Genetic Analyzer (PE Applied Biosystems) was used, and a detail of a method of preparing a sample, and a method of using an instrument was according to a manual attached to the present apparatus. For a vector DNA as a sample, a DNA prepared by an alkali extracting method was used, this was column-purified with GFXDNA Purification Kit (Amersham Biosciences), a DNA concentration was measured with a spectrophotometer Ultrospec 3000 (Amersham Biosciences), and the DNA was used.

### Example 2

(Preparation of Transformed Yeast)

A yeast W303-1a strain (haploid strain, TRP1 mutation, LEU2 mutation, URA3 mutation) which is a host is a strain deficient in the tryptophan synthesizing ability, the leucine synthesizing ability and the uracil synthesizing ability. This strain was cultured in 5 ml of a SD culturing solution at 30° C. to a logarithmic proliferation phase (OD₆₆₀ nm=0.8). From this, a competent cell was prepared using Frozen-EZ Yeast Transformation II kit (manufactured by ZYMO Research). According to a protocol attached to the kit, the chromosome introduction-type vector pBS-GAL1p-GFP constructed by the aforementioned Example was treated with a restriction enzyme XbaI, and this was introduced into this competent cell. This transformation sample was washed, dissolved in 100 μl of sterile water, and smeared on a tryptophan selection medium, and a transformant was selected under standing culture at 30° C.

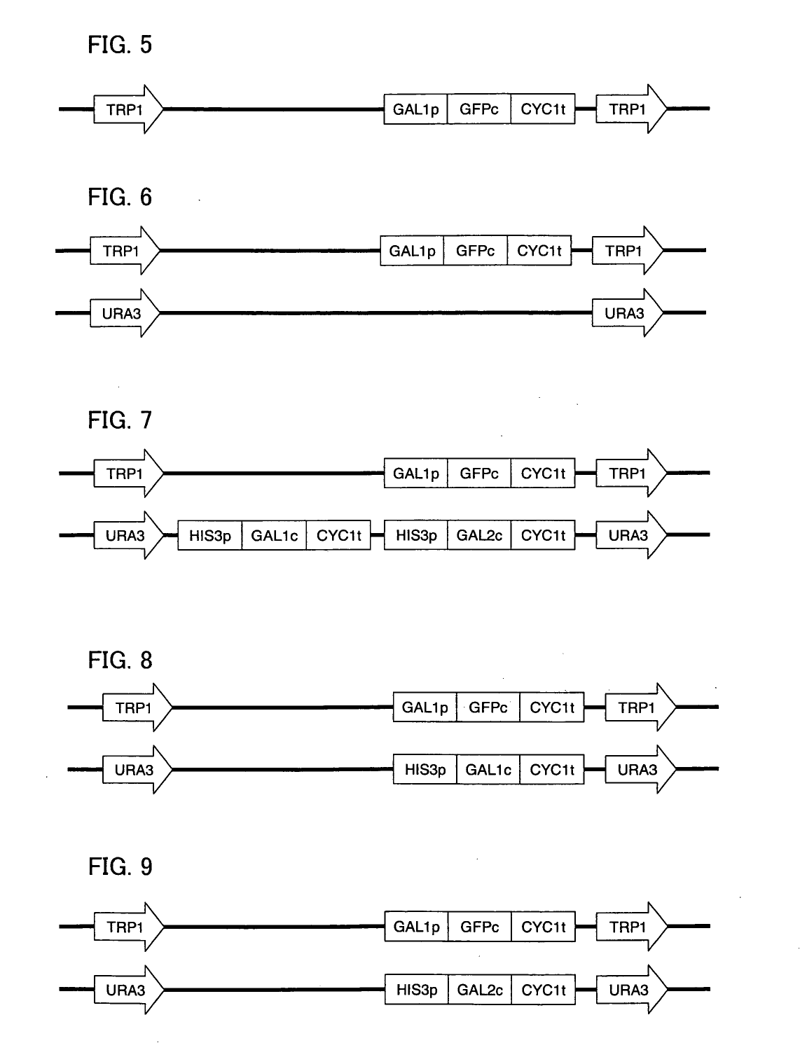

Each of the resulting colonies was isolated again on a new tryptophan selection medium, and a strain stably retaining the growing ability was adopted as a transformation candidate strain. Then, these candidate strains were cultured in 2ml of a YPD culturing solution overnight and, by using this together with a genomic DNA preparing kit, and Gen Torukun TM-for yeast-(manufactured by Takara Bio Inc.), a genomic DNA was prepared. PCR analysis was performed using the prepared each genomic DNA as a template, and one for which the presence of an introduced gene had been confirmed was designated as a transformed strain 1. A structure of an introduced gene in a chromosome in a transformed strain 1 is shown in FIG. 5.

Using the transformed strain 1, a competent cell was prepared by the same method as that described above. Each of the chromosome introduction-type vectors pBS-URA3 g, pBS-HIS3p-GAL2, pBS-HIS3p-GAL1, and pBS-HIS3p-GAL1-HIS3p-GAL2 constructed in the aforementioned Example was treated with a restriction enzyme NcoI, and this was introduced into this competent cell. This transformation sample was washed, dissolved in 100 μl of sterile water, smeared on an uracil selection medium, a transformant was selected while each was standing-cultured at 30° C., an introduced gene was confirmed by the same method as that described above, and ones for which the presence or the absence had been confirmed were designated as transformed strains 2 to 5. A construction of an introduced gene in a chromosome in these transformed yeast strains is shown in Table 1, and a structure is shown in FIG. 6 to FIG. 9.

TABLE 1

| | | Subject to be | | |
|---|---|---|---|---|
| | Subject to be | galactose- | Presence of | |
| Kind of | constitutively | inducibly | expression | |
| transformed | expressed by | expressed by | of GAL4 by | |
| strain | HIS3p | GAL1p | GAL10p | Examples |
| 2 | None | GFP | None | Control |
| | | | | Example |
| 3 | GAL2 | GFP | None | Example |
| 4 | GAL1 | GFP | None | Example |
| 5 | GAL1 + GAL2 | GFP | None | Example |
| 6 | None | GFP | Present | Comparative |
| | | | | Example |
| 7 | GAL1 + GAL2 | GFP | Present | Example |
| 8 | GAL1 + GAL2 | GFP | None | Example |

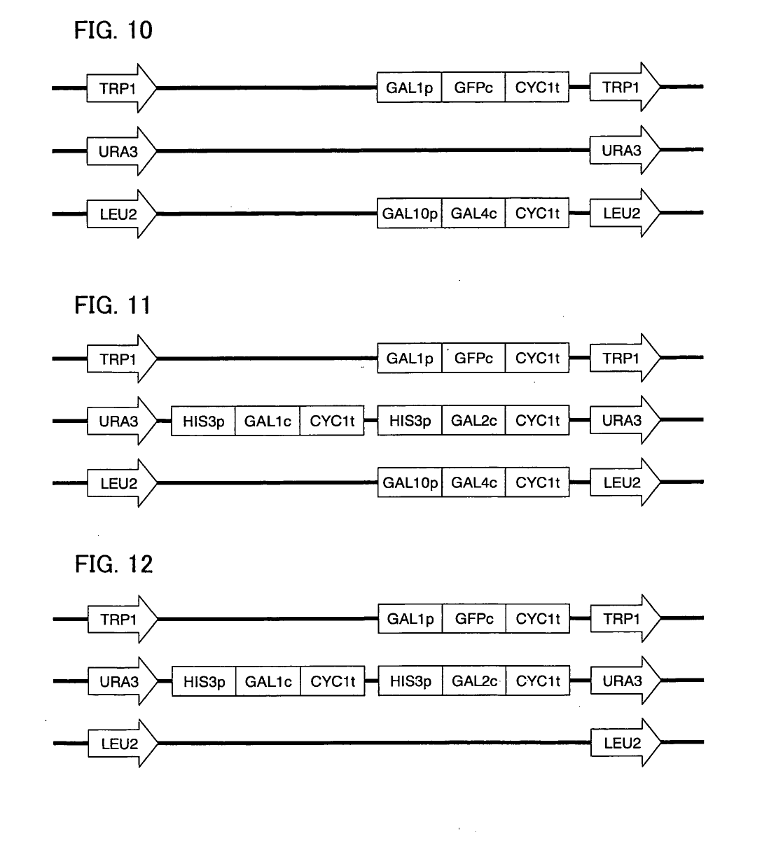

Using transformed strains 2 and 5, competent cells were prepared by the same method as that described above. The chromosome introduction-type vector pBS-GAL10p-GAL4 constructed in the aforementioned Example was treated with a restriction enzyme EcoRI, and this was introduced in these competent cells. Separately, pBS-LEU2 g was treated with a restriction enzyme EcoRI, and this was introduced into a competent cell of the transformant strain 5. These transformation samples were washed, dissolved in 100 μl of sterile water, smeared on a leucine selection medium, a transformant was selected while each was standing-cultured at 30° C., an introduced gene was confirmed by the same method as that described above, and ones for which the presence could be confirmed were designated as transformed strains 6 to 8. A construction of an introduced gene in a chromosome in each transformed yeast strain is shown in Table 1, and a structure is shown in FIGS. 10 to 12.

### Example 3

Testing of galactose Inducing Performance by Fermentation Test

These transformed strains 2 to 8 were cultured in the presence of glucose (addition of no galactose) under the following condition, thereafter, a medium was exchanged, strains were cultured in the presence of galactose (0.05 g/l to 20 g/l, addition of no glucose), the produced protein was extracted, and an amount of a GFP protein was assessed by Western blotting.

(Culturing Condition)

Each transformed strain was cultured in 5 ml of a SD culturing solution (glucose 2%) at 30° C. overnight. 100 μl of this pre-cultured solution was seeded in 10 ml of a SD culturing solution (glucose 2%), having OD₆₆₀ₙₘ at 30° C. =1.0 to 1.2 was adopted at 0 hour, 1 ml of a culturing solution was extracted and the cells were collected by centrifugation, and stored at −20° C. A remaining culturing solution was centrifuged to collect the cells, 8 ml of a SC medium (galactose concentration: 0.05 g/l to 20 g/1) was added to suspend it, and this was cultured at 30° C. for 0 to 4 hours under the aerobic condition (L-shaped test tube, 70 rpm (small-type shaking culturing device (ADVANTEC)). 1 ml of a culturing solution was taken with time (1h, 2h and 4h), centrifuged to collect the cells, which were stored at −20°C.

(Protein Extraction)

The cells which had been stored at −20° C. were thawed, to 6 μl of it were added 50 mM PMSF and 50μl Y-PER Yeast Protein Extraction Reagent (PIERCE), and this was treated at room temperature for 20 minutes while mildly shaken. This was centrifuged at 15,000 rpm and 40° C. for 15 minutes, the supernatant was transferred to 1.5 ml of another tube, and this was used as a protein solution. This protein solution was ⅓ diluted, and an absorbance of OD595 was measured with a spectrophotometer Ultraspec 3000 (Amersham Biosciences) employing a BSA solution and using QuickStart protein assay kit (BIO-RAD), and a protein concentration was determined.

(Western Blotting)

A 12% acrylamide gel was prepared, and SDS-polyacrylamide gel electrophoresis was performed at a constant voltage of 100 V using a Tris-Glycine buffer. A gel was immersed in a Towbin transcription buffer (25 mM Tris, 192 mM Glycine, 20% Methanol, pH 8.3) for 20 minutes to equilibrate this. Using a trans blot SD cell (BIO-RAD), a protein in a gel was transcribed to the already equilibrated protein transcription membrane Hybond-P (Amersham Biosciences) with the aforementioned transcription buffer at a constant voltage of 15 V for 40 minutes. The membrane was mildly rinsed with water, immersed in a 1×TBS buffer containing 2% BSA, and shaken at room temperature overnight to perform blocking.

An anti-autofluorescent protein, monoclonal antibody Anti-AFP, mAb (11E5) (manufactured by Wako Pure Chemical Industries, Ltd.) as a primary antibody was 1:1000-fold diluted in a 1×TBS buffer. A membrane was immersed in this solution, and this was shaken at room temperature for 2 hours. A membrane was washed with a 1×TBS buffer containing 0.1% Tween20 (BIO-RAD) at room temperature for 10 minutes while shaking. A buffer was exchanged, and this washing procedure was repeated three times.

Then, Anti mouse-IgG (peroxidase-linked) as a secondary antibody contained in ECLPlus Western blotting reagent pack (Amersham Biosciences) was 1:10000-fold diluted with a 1×TBS buffer. A membrane was immersed in this solution, and this was shaken at room temperature for 2 hours. A membrane was washed with a 1×TBS buffer containing 0.1% Tween20 (BIO-RAD) at room temperature for 10 minutes while shaking. A buffer was exchanged, and this washing procedure was repeated three times.

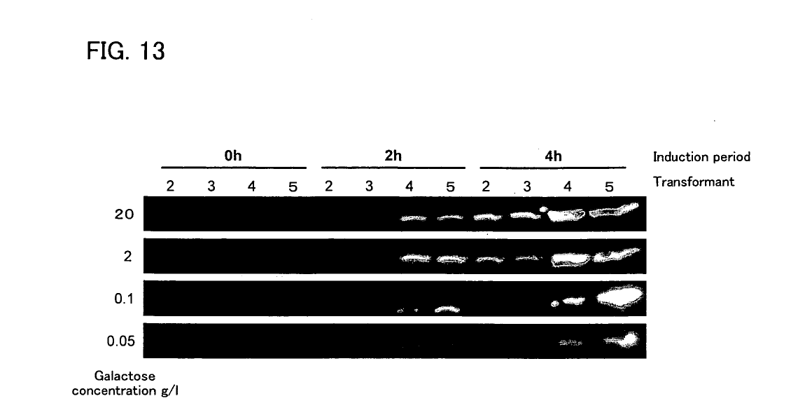

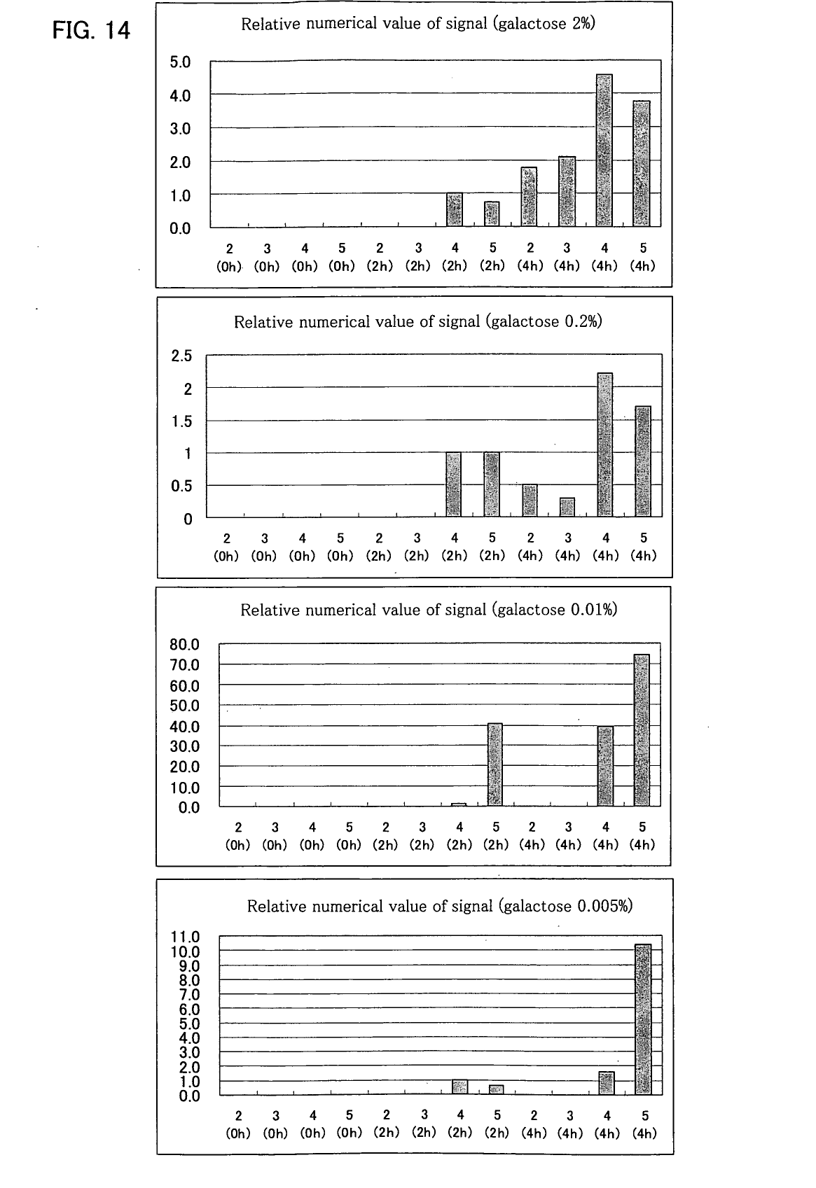

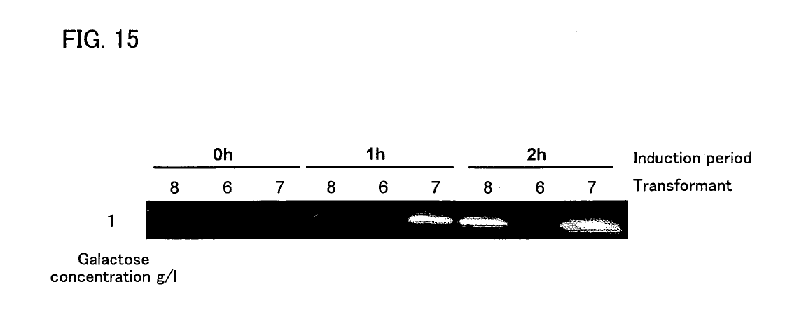

According to a protocol of the ECLPlus kit, chemiluminescence was performed (2%, 0.2%, 2 min; 0.01%, 0.005%, 10 min), and a signal was detected with a chemiluminescence detector (Aisin Seiki Co., Ltd.), to obtain image data and numerical value data of a light emitting intensity. Image data of transformed strains 2 to 5 are shown in FIG. 13, and a graph based on the resulting measured values is shown in FIG. 14. A light emitting intensity in these graphs is a relative numerical value data by every membrane, and a ratio is shown, letting a light emitting intensity after culturing of a transformed strain 4 in a membrane on a galactose inducing medium for 2 hours to be 1. In addition, image data of transformed strains 6 to 8 are shown in FIG. 15.

As shown in FIG. 13 and FIG. 14, a transformed strain 4 constitutively expressing Gal1 with a HIS3 promoter, and a transformed strain 5 constitutively expressing Gal1 and Gal2 with a HIS3 promoter showed the strongest light emitting intensity at all galactose concentrations. In addition, a transformed strain 3 expressing only Gal2 with a HIS3 promoter showed a higher emitting intensity than that of a transformed strain 2 as a control when a galactose concentration was 20 g/l. From this, it was seen that, by constitutively expressing either of Gal1, Gal3 and Gal2 which are a protein assisting transcription activation by Gal4, GFP can be galactose-inducibly expressed effectively.

In addition, when a galactose concentration was 20 g/l and 2 g/l, a transformed strain 4 constitutively expressing only Gal1 showed a higher emitting intensity than a transformed strain 5 constitutively expressing Gal1 and Gal2. On the other hand, when a galactose concentration is 0.1 g/l and 0.05 g/l, a transformed strain 5 showed a considerably higher emitting intensity than a transformed strain 4. That is, in the presence of galactose at a relatively high concentration, between transformed strains 4 and 5, a transformed strain 4 constitutively expressing only Gal1 is rather predominant, but responsiveness of galactose induction is not considerably different, while when a galacatose concentration is lowered, these responsivenesses are reversed, and as a concentration was lower, a difference in responsiveness became remarkably greater. From the forgoing, it was seen that, when one tries to obtain effective responsiveness at a relatively low concentration of galactose, it is preferable to use a combination of Gal1 and Gal2. In addition, it was seen that the synergistic action of Gal2 (galactopermease) with Gal1 is potentiated when a galactose concentration is low. Further, it was seen that constitutive expression of only Gal2 by a HIS3 promoter is effective when a galactose concentration is high to some extent, and increase in the effect due to stronger constitutive expression can be expected.

In addition, as shown in FIG. 15, both of transformed strains 7 and 8 constitutively expressing Gal1 and Gal2 produce GFP earlier and at a larger amount than a transformed strain 6 galactose-inducibly expressing Gal4. At least in 2 hours, a significant signal could not be detected in a transformed strain 6. In addition, particularly, in a transformed strain 7 constitutively expressing Gal1 and Gal2 and, at the same time, galactose-inducibly expressing Gal4, better responsiveness was exhibited than a transformed strain 6.

From the forgoing, it was seen that, only by galactose-inducibly expressing Gal4, a galactose induction system cannot be induced responsively. And, it was seen that, by constitutively expressing an assisting protein such as Gal1 and Gal2 and, at the same time, galactose-inducibly expressing Gal4, the synergistic effect is exhibited.

The present application claims the benefit of priority from Japanese Patent Application No. 2005-285048 filed on Sep. 29, 2005, the contents of which are incorporated herein by reference in their entirety.

### SEQUENCE LISTING FREE TEXT

SEQ ID Nos. 1 to 26: primer

What is claimed is:

1. A transformant having a galactose induction system, and retaining a coding region encoding an assisting protein assisting transcription activation by a protein having the Gal4 activity under control of a constitutive promoter on a host chromosome or outside a host chromosome.

2. The transformant according to claim 1, wherein the assisting protein includes a protein having the Gal80 binding activity and/or the galactokinase activity.

3. The transformant according to claim 2, wherein the assisting protein is selected from a protein having the Gal1 activity and a protein having the Gal3 activity.

4. The transformant according to claim 3, wherein the protein having the Gal80 binding activity is a protein having the Gal1 activity.

5. The transformant according to claim 1, wherein the assisting protein includes a protein having the galactose transporter activity.

6. The transformant according to claim 5, wherein the assisting protein is a protein having the Gal2 activity.

7. The transformant according to claim 1, wherein the assisting protein includes a protein having the Gal80 binding activity and/or the galactokinase activity, and a protein having the galactose transporter activity.

8. The transformant according to claim 7, wherein the assisting protein includes a protein having the Gal1 activity and a protein having the Gal2 activity.

9. The transformant according to claim 1, wherein the constitutive promoter is selected from a HIS3 promoter, a TDH3 promoter and an ADH1 promoter.

10. The transformant according to claim 1, wherein the transformant retains a coding region encoding a protein having the Gal4 activity under control of a galactose-inducible promoter on a host chromosome or outside a host chromosome.

11. The transformant according to claim 1, wherein the transformant retains a coding region encoding an endogenous or exogenous desired protein under control of the galactose-inducible promoter.

12. The transformant according to claim 10, wherein the galactose-inducible promoter is selected from the group consisting of a GAL1 promoter, a GAL2 promoter, a GAL3 promoter, a GAL4 promoter, a GAL5 promoter, GAL7 promoter, a GAL10 promoter and a MEL1 promoter.

13. The transformant according to claim 10, wherein the galactose-inducible promoter is an endogenous promoter or an exogenous promoter other than a galactose metabolism gene, which is provided with a Gal4 binding site on an upstream site and can be activated by a protein having the Gal4 activity.

14. The transformant according to claim 13, wherein the exogenous promoter or the endogenous promoter is selected from a CYC1 promoter, a CUP1 promoter, and a HOR7 promoter.

15. The transformant according to claim 1, wherein the host cell is yeast.

16. The transformant according to claim 15, wherein the yeast is selected from Saccharomyces cerevisiae, Saccharomyces pombe, Pichia pastris, Kluyveromyces lactis and Candida albicans.

17. A nucleic acid construct comprising:
a constitutive promoter, and
a coding region encoding an assisting protein assisting transcription activation by a protein having the Gal4 activity ligated under control of the constitutive promoter.

18. The nucleic acid construct according to claim 17, wherein the assisting protein includes a protein having the Gal80 binding activity and/or the galactokinase activity.

19. The nucleic acid construct according to claim 18, wherein the assisting protein is selected from a protein having the Gal1 activity and a protein having the Gal3 activity.

20. The nucleic acid construct according to claim 17, wherein the assisting protein includes a protein having the galactose transporter activity.

21. The nucleic acid construct according to claim 20, wherein the assisting protein is a protein having the Gal2 activity.

22. A nucleic acid construct comprising:
a first constitutive promoter,
a first coding region encoding a protein having the Gal80 binding activity and/or a protein having the galactokinase activity ligated under control of the first constitutive promoter,
a second constitutive promoter, and
a second coding region encoding a protein having the galactose transporter activity ligated under control of the second constitutive promoter.

23. An expression vector comprising a nucleic acid construct according claim 17.

24. The expression vector according to claim 23, being a plasmid or a virus.

25. A host cell retaining an expression vector according to claim 23.

26. A method of culturing a transformant, comprising:
a culturing step of culturing a transformant according to claim 11 in a medium not substantially containing galactose.

27. The culturing method according to claim 26, further comprising a culturing step of culturing the transformant in the presence of galactose to such an extent that glucose is not substantially contained and the galactose induction system of the transformant can be expressed.

28. A method of expressing a gene, comprising:
a first culturing step of culturing a transformant according to claim 11 in a medium not substantially containing galactose, and
a second culturing step of culturing the transformant in the presence of galactose to such an extent that glucose is not substantially contained and the galactose induction system of the transformant can be expressed, to express the desired protein.

29. The expression method according to claim 28, wherein an initial concentration of galactose in the second culturing step is less than 20 g/l.

30. The expression method according to claim 28, wherein the second culturing step is initiated by exchanging a medium with a medium containing galactose.

31. A process for producing a substance using a transformant, comprising:
a step of recovering a desired protein expressed by a gene expression method according to claim 28, or a metabolism product of the protein.
